SEQUENCE LISTING (1 sequence)

SEQ ID NO: 1 - DNA, 12 bp, synthetic construct
  source 1..12: mol_type = other DNA
tatggtcgtcga

(19) United States
(12) Patent Application Publication
(10) Pub. No.: US 2024/0153583 A1
(43) Pub. Date: May 9, 2024

(54) METHODS AND SYSTEMS FOR INCREASING SEQUENCING QUALITY

(71) Applicant: Ultima Genomics, Inc., Fremont, CA (US)

(72) Inventors: Yoav ETZIONI, Tel Aviv (IL); Edward PERELMAN, Lehavim (IL)

(21) Appl. No.: 18/417,825

(22) Filed: Jan. 19, 2024

Related U.S. Application Data
(63) Continuation of application No. PCT/US2022/074056, filed on Jul. 22, 2022.
(60) Provisional application No. 63/203,479, filed on Jul. 23, 2021.

(51) Int. Cl. G16B 30/00; G16B 40/30

(52) U.S. Cl. CPC G16B 30/00; G16B 40/30

(57) ABSTRACT

Described herein are methods and systems for improving nucleic acid sequencing read quality. An exemplary method comprises receiving, at one or more processors, sequencing data comprising a plurality of sequencing reads; filtering the sequencing data, using the one or more processors, to remove sequencing reads for which an absence of an incorporated nucleotide was detected at three or more consecutive sequencing flow steps, thereby generating filtered sequencing data; determining, using the one or more processors, for each sequencing flow step of each sequencing read, a read quality metric based on one or more homopolymer probability values other than a highest homopolymer probability value; and trimming the terminus of one or more sequencing reads in the sequencing data based on the read quality metrics for a respective sequencing read, thereby generating trimmed sequencing data.

### CROSS-REFERENCE TO RELATED APPLICATION

This application claims the priority benefit of U.S. Provisional patent application Ser. No. 63/203,479, filed Jul. 23, 2021; the contents of which are incorporated herein by reference in its entirety.

### REFERENCE TO AN ELECTRONIC SEQUENCE LISTING

The contents of the electronic sequence listing (165272001040SEQLIST.xml; Size: 1,903 bytes; and Date of Creation: Jul. 20, 2022) is herein incorporated by reference in its entirety.

### FIELD OF THE INVENTION

Described herein are methods and systems for improving nucleic acid sequencing read quality.

### BACKGROUND

Next-generation sequencing (NGS), or massively parallel sequencing, allows for the generation of large amounts of sequencing data used to determine the sequence of target nucleic acid molecules. Many NGS methods rely on the extension of a primer strand and the incorporation of labeled nucleotide bases, which can be detected and analyzed to determine the sequence of a template target nucleic acid. Resulting sequencing reads may be mapped to a reference sequence and, based on differences between the sequencing reads and the reference sequences at the mapped loci, variants may be called.

As the length of a sequencing read increases (e.g., as more flow steps are performed to extend the sequencing read), the quality of the sequencing read deteriorates. This may be attributable to, for example, lagging or failed primer extension strands within a sequencing colony that accumulate during the sequencing run. As a result of this sequencing read quality deterioration, the sequencing read can become inaccurate especially in later-identified portions, and thus cannot be aligned to a reference read. In some sequencing runs, a significant percentage of the sequencing reads are unusable, for example because they cannot be accurately aligned to a reference sequence. Because variant calling and other downstream processes often rely on accurate sequence alignments, poor sequencing read quality presents a substantial hurdle in accurately assessing nucleic acid molecules.

### BRIEF SUMMARY OF THE INVENTION

Described herein are methods and systems for increasing sequencing read quality by generating filtered and/or trimmed sequencing data. The methods thus provide sequencing reads that are better suited for alignment to a reference sequence (e.g., a reference genome) compared to sequencing reads that have not been improved by the process. As an added benefit, the resulting sequencing data can be stored on a computer-readable medium with a reduced file size compared to pre-processed sequencing data. The reduced file sizes require less computer storage space, thus leading to improved usage and management of computer memory. The smaller files can be faster to process in downstream tasks (e.g., variant calling), resulting in a more efficient use of computer processing power. Further, the smaller files contain cleaner, better-structured data, thus improving the analysis capability of downstream tasks. Thus, embodiments of the present disclosure improve the functioning of computer systems and sequencing systems. Through novel data structures and logics, embodiments of the present disclosure provide improved memory usage, improved memory management, and improved processing to support the high-throughput and high-precision requirements of the flow sequencing method to provide high-quality sequencing reads.

An exemplary method for increasing sequencing read quality comprises: receiving, at one or more processors, sequencing data comprising a plurality of sequencing reads generated by extending a sequencing primer through a region of interest in a target nucleic acid molecule using a plurality of sequencing flow steps, each sequencing flow step comprising combining a hybrid with nucleotides, the hybrid comprising the sequencing primer and a nucleic acid molecule comprising the region of interest, wherein at least a portion of the nucleotides are labeled, and detecting the presence or absence of an incorporated nucleotide; filtering the sequencing data, using the one or more processors, to remove sequencing reads for which an absence of an incorporated nucleotide was detected at three or more consecutive sequencing flow steps, thereby generating filtered sequencing data; determining, using the one or more processors, for each sequencing flow step of each sequencing read, a read quality metric based on one or more homopolymer probability values other than a highest homopolymer probability value; and trimming the terminus of one or more sequencing reads in the sequencing data based on the read quality metrics for a respective sequencing read, thereby generating trimmed sequencing data.

In some embodiments, the method further comprises generating the sequencing data.

In some embodiments, the method further comprises calling, using the one or more processors, one or more genetic variants using the trimmed sequencing data.

In some embodiments, the method further comprises trimming a known adapter sequence, or a portion thereof, from one or more sequencing reads in the sequencing data.

In some embodiments, the read quality metric for each sequencing flow step of each sequencing read is based on a second highest homopolymer probability value.

In some embodiments, trimming the terminus of the one or more sequencing reads in the sequencing data based on the read quality metric, thereby generating the trimmed sequencing data, comprises, for each sequencing read: determining a read quality metric moving average for the sequencing flow steps; selecting a sequencing flow step, wherein the selected sequencing flow step is the nth sequencing flow step having a moving average above a predetermined threshold, wherein n is a predefined number; and trimming at least a portion of the sequencing read comprising the selected sequencing flow step.

In some embodiments, a predetermined number of consecutive sequencing flow steps prior to the selected sequencing flow step are trimmed.

In some embodiments, the predetermined number of consecutive sequencing flow steps is a multiple of four.

In some embodiments, the method further comprises storing the trimmed sequencing data in a non-transitory computer readable medium.

In some embodiments, the method further comprises aligning sequencing reads in the trimmed sequencing data to a reference sequence. In some embodiments, the reference sequence is a reference genome.

In some embodiments, at least a predetermined percentage of sequencing reads in the trimmed sequencing data are aligned to the reference sequence. In some embodiments, the reference sequence is a reference genome.

In some embodiments, the predetermined percentage is about 70%, about 75%, about 80%, about 85%, about 90%, about 95%, about 99%, or about 100%.

In some embodiments, the nucleotides are non-terminating nucleotides.

An exemplary system comprises: one or more processors; and a non-transitory computer readable medium storing one or more programs which, when executed by the one or more processors, are configured to: receive, at the one or more processors, sequencing data comprising a plurality of sequencing reads generated by extending a sequencing primer through a region of interest using a plurality of sequencing flow steps, each sequencing flow step comprising combining a hybrid with nucleotides, the hybrid comprising the sequencing primer and a nucleic acid molecule comprising the region of interest, wherein at least a portion of the nucleotides are labeled, and detecting the presence or absence of an incorporated nucleotide; filter the sequencing data, using the one or more processors, to remove sequencing reads for which an absence of an incorporated nucleotide was detected at three or more consecutive sequencing flow steps, thereby generating filtered sequencing data; determine, using the one or more processors, for each flow step of each sequencing read, a read quality metric based on one or more homopolymer probability values other than a highest homopolymer probability value; and trim the terminus of one or more sequencing reads in the sequencing data based on the read quality metrics for a respective sequencing read, thereby generating trimmed sequencing data.

In some embodiments, the system further comprises a sequencer configured to generate the sequencing data.

In some embodiments, the one or more programs, when executed by the one or more processors, are further configured to call, using the one or more processors, one or more genetic variants using the trimmed sequencing data.

In some embodiments, the one or more programs, when executed by the one or more processors, are further configured to trim a known adapter sequence, or a portion thereof, from one or more sequencing reads in the sequencing data.

In some embodiments, the read quality metric for each sequencing flow step of each sequencing read is based on a second highest homopolymer probability value.

In some embodiments, trimming the terminus of the one or more sequencing reads in the sequencing data based on the read quality metric, thereby generating the trimmed sequencing data, comprises, for each sequencing read: determining a read quality metric moving average for the sequencing flow steps; selecting a sequencing flow step, wherein the selected sequencing flow step is the nth sequencing flow step having a moving average above a predetermined threshold, wherein n is a predetermined number; and trimming at least a portion of the sequencing read comprising the selected sequencing flow step.

In some embodiments, a predetermined number of consecutive sequencing flow steps prior to the selected sequencing flow step are trimmed.

In some embodiments, the predetermined number of consecutive sequencing flow steps is a multiple of four.

In some embodiments, the one or more programs, when executed by the one or more processors, are further configured to store the trimmed sequencing data in the non-transitory computer readable medium.

In some embodiments, the one or more programs, when executed by the one or more processors, are further configured to align sequencing reads in the trimmed sequencing data to a reference sequence. In some embodiments, the reference sequence is a reference genome.

In some embodiments, at least a predetermined percentage of sequencing reads in the trimmed sequencing data are aligned to the reference sequence. In some embodiments, the reference sequence is a reference genome.

In some embodiments, the predetermined percentage is about 70%, about 75%, about 80%, about 85%, about 90%, about 95%, about 99%, or about 100%.

In some embodiments, the nucleotides are non-terminating nucleotides.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The invention will now be described, by way of example only, with reference to the accompanying drawings, in which:

FIG. 1 illustrates an exemplary flow sequencing method that can be used to generate sequencing data, in accordance with some embodiments.

FIG. 2A illustrates an exemplary summary of detected signals after a number of exemplary flow cycles are performed, in accordance with some embodiments.

FIG. 2B illustrates an exemplary process for determining a preliminary sequence, in accordance with some embodiments.

FIG. 3 illustrates an exemplary method for increasing sequencing read quality, in accordance with some embodiments.

FIG. 4A illustrates an exemplary plurality of sequencing reads, in accordance with some embodiments.

FIG. 4B illustrates a filtered set of sequencing reads, in accordance with some embodiments.

FIG. 4C illustrates a filtered and trimmed set of sequencing reads, in accordance with some embodiments.

FIG. 5A illustrates that three consecutive sequencing flow steps cannot all yield a signal of 0 indicating an absence of an incorporated nucleotide, in accordance with some embodiments.

FIG. 5B illustrates that three consecutive sequencing flow steps cannot all yield a signal of 0 indicating an absence of an incorporated nucleotide, in accordance with some embodiments.

FIG. 5C illustrates that three consecutive sequencing flow steps cannot all yield a signal of 0 indicating an absence of an incorporated nucleotide, in accordance with some embodiments.

FIG. 6 illustrates the read quality metrics for an exemplary sequencing read, in accordance with some embodiments.

FIG. 7A illustrates that quality issues may occur to an increasing percentage of reads as the number of flow steps increase, in accordance with some embodiments.

FIG. 7B illustrates a plurality of exemplary sequencing reads, in accordance with some embodiments.

FIG. 8A illustrates that quality issues may occur to an increasing percentage of reads as the number of flow steps increase, in accordance with some embodiments.

FIG. 8B illustrates a plurality of exemplary sequencing reads, in accordance with some embodiments.

FIG. 9 illustrates exemplary results of a method for increasing sequencing read quality, in accordance with some embodiments

FIG. 10 illustrates an exemplary electronic device, in accordance with some embodiments.

FIG. 11 illustrates the percentage of reads trimmed or filtered as the number of flow steps increase, in accordance with some embodiments.

FIG. 12 illustrates the read length distribution for sequencing reads that have been trimmed or filtered, in accordance with some embodiments.

FIG. 13A illustrates an example block diagram of sequencing read data sets in accordance with embodiments described herein.

FIG. 13B illustrates another example block diagram of sequencing read data sets in accordance with embodiments described herein.

FIG. 13C illustrates another example block diagram of sequencing read data sets in accordance with embodiments described herein.

FIG. 13D illustrates another example block diagram of sequencing read data sets in accordance with embodiments described herein.

### DETAILED DESCRIPTION OF THE INVENTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown but are to be accorded the scope consistent with the claims.

Disclosed herein are methods, electronic devices, systems, and non-transitory storage media for biological sample processing and/or analysis. Nucleic acid sequencing read quality can be improved by generating trimmed sequencing data according to the methods described herein, which may be implemented using the described systems. Raw sequencing data can include portions, particularly the later-sequenced terminus of the sequencing read, that are of poor read quality. By improving the sequencing read quality of the sequencing data, the sequencing reads can more reliably be aligned to a reference sequence (e.g., a reference genome). Further, resulting file sizes may be smaller, which allows for more convenient data storage (e.g., on a non-transitory computer readable medium) or downstream processing.

Sequencing data processed using the described methods may be generated using a flow sequencing method. As further described herein, flow sequencing is a sequencing methodology that relies on extending a primer through a region of interest using a plurality of sequencing flow steps. The sequencing flow steps each comprise combining a hybrid (which includes a sequencing primer and a nucleic acid molecule comprising the region of interest) with nucleotides. At least a portion of the nucleotides are labeled, and the presence or absence of an incorporated nucleotide is detected during the sequencing flow step.

Sequencing read quality can be increased by receiving (for example, by one or more processors) sequencing data comprising a plurality of sequencing reads generated by the flow sequencing method; filtering the sequencing data (for example, using the one or more processors) to remove sequencing reads for which an absence of an incorporated nucleotide was detected at three or more consecutive sequencing flow steps, thereby generating filtered sequencing data; determining (for example, using the one or more processors) for each flow step of each sequencing read, a read quality metric based on one or more homopolymer probability values other than a highest homopolymer probability value; and trimming the terminus of one or more sequencing reads in the sequencing data based on the read quality metrics for a respective sequencing read, thereby generating trimmed sequencing data.

The trimmed sequencing data may be stored on a non-transitory computer readable storage medium. Additionally or alternatively, the trimmed sequencing data may be used in a downstream process. For example, the trimmed sequencing reads in the trimmed sequencing data may be aligned to a reference sequence (e.g., a reference genome or portions thereof). In some embodiments, one or more genetic variants may be called using the trimmed sequencing data set, for example based on a comparison of the trimmed sequencing reads to the reference sequence.

Embodiments of the present disclosure can produce smaller files of sequencing reads for downstream tasks (e.g., variant calling). The smaller files require less computer storage space, thus leading to improved usage and management of computer memory. The smaller files can be faster to process in downstream tasks, resulting in a more efficient use of computer processing power. Further, the smaller files contain cleaner, better-structured data, thus improving the analysis capability of downstream tasks. Thus, embodiments of the present disclosure improve the functioning of computer systems and sequencing systems. Through novel data structures and logics, embodiments of the present disclosure provide improved memory usage, improved memory management, and improved processing to support the high-throughput and high-precision requirements of the flow sequencing method to provide high-quality sequencing reads.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

“Expected sequencing data” refers to sequencing data one would expect if the sequence of a polynucleotide used to generate a coupled sequencing read pair, or the sequence of a region of said polynucleotide, matches a reference sequence.

A “flow order” refers to the order of separate nucleotide flows used to sequence a nucleic acid molecule using non-terminating nucleotides. A flow order may have any number of nucleotide flows. A flow order may be expressed as a one-dimensional matrix or linear array of bases corresponding to the identities of, and arranged in chronological order of, the nucleotide flows provided to the sequencing reaction space:

(e.g., [A-T-G-C-A-T-G-C-A-T-G-A-T-G-A-T-G-A-T-G-C-A-T-G-C]). Such a one-dimensional matrix or linear array of bases in the flow order may also be referred to herein as a “flow space.” Each entry in flow space (e.g., each element in the one-dimensional matrix or linear array) indicates a flow position. A “flow position” refers to the sequential position of a given separate nucleotide flow during the sequencing process. The flow order may be divided into cycles of repeating units (i.e., a “flow cycle”), and the flow order of the repeating units is termed a “flow-cycle order.” A flow cycle may be expressed as a one-dimensional matrix or linear array of an order of bases corresponding to the identities of, and arranged in chronological order of, the nucleotide flows provided within the sub-group of contiguous flow(s) (e.g., [A-T-G-C], [A-A-T-T-G-G-C-C], [A-T], [A/T-A/G], [A-A], [A], [A-T-G], etc.). A flow cycle may have any number of nucleotide flows. A given flow cycle may be repeated one or more times in the flow cycle, consecutively or non-consecutively. For example, where [A-T-G-C] is identified as a 1st flow cycle, and [A T G] is identified as a 2nd flow cycle, the flow order of [A-T-G-C-A-T-G-C-A-T-G-A-T-G-A-T-G-A-T-G-C-A-T-G-C] may be described as having a flow-cycle order of [1st flow cycle; 1st flow cycle; 2nd flow cycle; 2nd flow cycle; 2nd flow cycle; 1st flow cycle; 1st flow cycle]. Alternatively or in addition, the flow-cycle order may be described as [cycle 1, cycle 2, cycle 3, cycle 4, cycle 5, cycle 6], where cycle 1 would be the 1st flow order, cycle 2 would be the 1st flow order, cycle 3 would be the 2nd flow cycle order, etc.

The term “homopolymer length” refers to a number of sequential identical nucleotides of a particular base type in a nucleic acid sequence at a given flow step. The homopolymer length may be 0, 1, 2, 3 or any other 0 or positive integer value. A “homopolymer length likelihood” refers to a statistical parameter indicative of a likelihood or confidence that a given homopolymer length at a particular flow step is the correct homopolymer length.

The terms “individual,” “patient,” and “subject” are used synonymously, and refers to an individual or entity from which a biological sample (e.g., a biological sample that is undergoing or will undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer, or cervical cancer) or an infectious disease. Alternatively or in addition, a subject may be known to have previously had a disease or disorder. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may have a given weight, height, body mass index, or other physical characteristic. A subject may have a given ethnic or racial heritage, place of birth or residence, nationality, disease or remission state, family medical history, or other characteristic. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

As used herein, the term “biological sample” generally refers to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. The biological sample can be a fluid, tissue, collection of cells (e.g., cheek swab), hair sample, or feces sample. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. Alternatively, the sample may be obtained from any other source including but not limited to blood, sweat, hair follicle, buccal tissue, tears, menses, feces, or saliva of a subject. The biological sample may be a tissue sample, such as a tumor biopsy. The tissue can be from an organ (e.g., liver, lung, or thyroid), or a mass of cellular material, such as, for example, a tumor. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively or in addition, nucleic acid molecules may not be included within cells (e.g., cell-free nucleic acid molecules). The biological sample may be a cell-free sample.

The term “cell-free sample,” as used herein, generally refers to a sample that is substantially free of cells (e.g., less than 10% cells on a volume basis). A cell-free sample may be derived from any source (e.g., as described herein). For example, a cell-free sample may be derived from blood, sweat, urine, or saliva. For example, a cell-free sample may be derived from a tissue or bodily fluid. A cell-free sample may be derived from a plurality of tissues or bodily fluids. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

The term “label,” as used herein, refers to a detectable moiety that is coupled to or may be coupled to another moiety, for example, a nucleotide or nucleotide analog. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease). In some embodiments, the label is a fluorophore.

The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide). The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). A “non-terminating nucleotide” is a nucleic acid moiety that can be attached to a 3′ end of a polynucleotide using a polymerase or transcriptase, and that can have another non-terminating nucleic acid attached to it using a polymerase or transcriptase without the need to remove a protecting group or reversible terminator from the nucleotide. Naturally occurring nucleic acids are a type of non-terminating nucleic acid. Non-terminating nucleic acids may be labeled or unlabeled.

A “nucleotide flow” refers to a set of one or more non-terminating nucleotides (which may be labeled or a portion of which may be labeled). The nucleotide flow may be provided to a sequencing reaction space in a temporally distinct instance of providing a nucleotide-containing reagent. For example, providing two flows may refer to (i) providing a nucleotide-containing reagent (e.g., an A-base containing solution) to a sequencing reaction space at a first time point and (ii) providing a nucleotide-containing reagent (e.g., a G-base containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of canonical base types (A, T, G, C; or U), e.g., 1, 2, 3, or 4 canonical base types.

A “short genetic variant” is used herein to describe a genetic polymorph (i.e., mutation) 10 consecutive bases in length or less (i.e., 10, 9, 8, 7, 6, 5, 4, 3, 2, or 1 base(s) in length). The term includes single nucleotide polymorphisms (SNPs), multi-nucleotide polymorphisms (MNPs), and indels 10 consecutive bases in length or less.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or deoxyribonucleic acids (DNA) or ribonucleotides or ribonucleic acids (RNA), or analogs thereof. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid molecule can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), or more. A nucleic acid molecule (e.g., polynucleotide) can comprise a sequence of four natural nucleotide bases: adenine (A); cytosine (C); guanine (G); and thymine (T) (uracil (U) for thymine (T) when the polynucleotide is RNA). A nucleic acid molecule may include one or more nonstandard nucleotide(s), nucleotide analog(s) and/or modified nucleotide(s).

The terms “reference genome” and “reference sequence,” as used herein, generally refer to a standardized genomic sequence or a portion thereof (e.g., any genome known in the art). A reference genome may be a representative example of a set of genes. In some instances, a reference genome is generalized to a species (e.g., Homo sapiens) and is determined from one or more assembled or partially assembled genome sequences of one or more individuals of said species. In some instances, a reference genome is specific to an individual of a species, and in such instances the reference genome may be determined from one or more assembled or partially assembled genome sequences from said individual. A reference genome may be any portion of a genomic nucleic acid sequence (e.g., a targeted panel of genes, one or more chromosomes, an entire genome of a species, etc.) that is used as a comparison for generated nucleic acid sequencing data (e.g., sequencing information generated according to sequencing methods described herein). Examples of human reference genomes include NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38). Additional reference genomes can be found online in the National Center for Biotechnology Information (NCBI) of the University of California, Santa Cruz (UCSC) genome browsers.

The term “sequencing,” as used herein, generally refers to a process for generating or identifying a sequence of a biological molecule, such as a nucleic molecule. Such sequence may be a nucleic acid sequence, which may include a sequence of nucleic acid bases. Sequencing may be single molecule sequencing or sequencing by synthesis, for example. Sequencing may be performed using template nucleic acid molecules immobilized on a support, such as a flow cell or one or more beads on a substrate as described herein. Examples of sequencing may be single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The FIGURES illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Generating Sequencing Data Using Flow Sequencing Methods

Sequencing data can be generated using a flow sequencing method that includes extending a primer hybridized to a template polynucleotide molecule according to a pre-determined flow cycle or flow order where, in any given flow position, a type of nucleotide base is accessible to the extending primer. More commonly, a single type of nucleotide base is used in any given sequencing flow, although in some variations, two or three different types of nucleotide bases may be used, which allows for a faster primer extension but may provide less sequencing data about the sequence region. At least some of the nucleotides of the particular base type can include a label, which upon incorporation of the labeled nucleotides into the extending primer renders a detectable signal. The resulting sequence by which such nucleotides are incorporated into the extended primer should be the reverse complement of the sequence of the template polynucleotide molecule. For example, sequencing data may be generated using a flow sequencing method that includes (i) extending a primer using labeled nucleotides and (ii) detecting the presence or absence of a labeled nucleotide incorporated into the extending primer. Flow sequencing methods may also be referred to as “natural sequencing-by-synthesis,” or “non-terminated sequencing-by-synthesis” methods. Exemplary methods are described in U.S. Pat. No. 8,772,473; published International application WO 2021/007495; published International application WO 2020/0227143; and published International application WO 2020/227137; each of which is incorporated herein by reference in its entirety. While the following description is provided in reference to flow sequencing methods, it is understood that other sequencing methods may be used to sequence all or a portion of the sequenced region.

Flow sequencing includes the use of nucleotides to extend the primer hybridized to the polynucleotide. Nucleotides of a given base type (e.g., A, C, G, T, U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added), although two or three different types of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

The nucleotides can be introduced at a determined order during the course of primer extension, which may optionally be further divided into cycles. Nucleotides are added stepwise, which allows incorporation of the added nucleotide to the end of the sequencing primer of a complementary base in the template strand is present. The cycles may have the same order of nucleotides and number of different base types or a different order of nucleotides and/or a different number of different base types. Solely by way of example, the order of a first cycle may be A-T-G-C and the order of a second cycle may be A-T-C-G. In some instances, the order of any cycle may be any permutation of the nucleotides A, G, C, and T (or U). Between the introductions of different nucleotides, unincorporated nucleotides may be removed, for example by washing the sequencing platform with a wash fluid.

A polymerase can be used to extend a sequencing primer by incorporating one or more nucleotides at the end of the primer in a template-dependent manner. In some embodiments, the polymerase is a DNA polymerase. The polymerase may be a naturally occurring polymerase or a synthetic (e.g., mutant) polymerase. The polymerase can be added at an initial step of primer extension, although supplemental polymerase may optionally be added during sequencing, for example with the stepwise addition of nucleotides or after a number of flow cycles. Exemplary polymerases include a DNA polymerase, an RNA polymerase, a thermostable polymerase, a wild-type polymerase, a modified polymerase, Bst DNA polymerase, Bst 2.0 DNA polymerase Bst 3.0 DNA polymerase, Bsu DNA polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, and SeqAmp DNA polymerase.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template polynucleotide can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiment, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

The sequencing data can be generated by sequencing the test nucleic acid molecule using non-terminating nucleotides provided in separate nucleotide flows according to a flow-cycle order. The sequencing data can include flow signals at flow positions that each corresponds to a flow of a particular nucleotide. Using this uniquely structured data set, the nucleic acid molecule (or molecules) can be analyzed in “flow space” rather than “basespace” (also referred to as “nucleotide space” or “sequence space”). The flow space data depend on additional information related to the flow-cycle order, which is not carried by basespace data. See, for example, published International application WO 2020/227137.

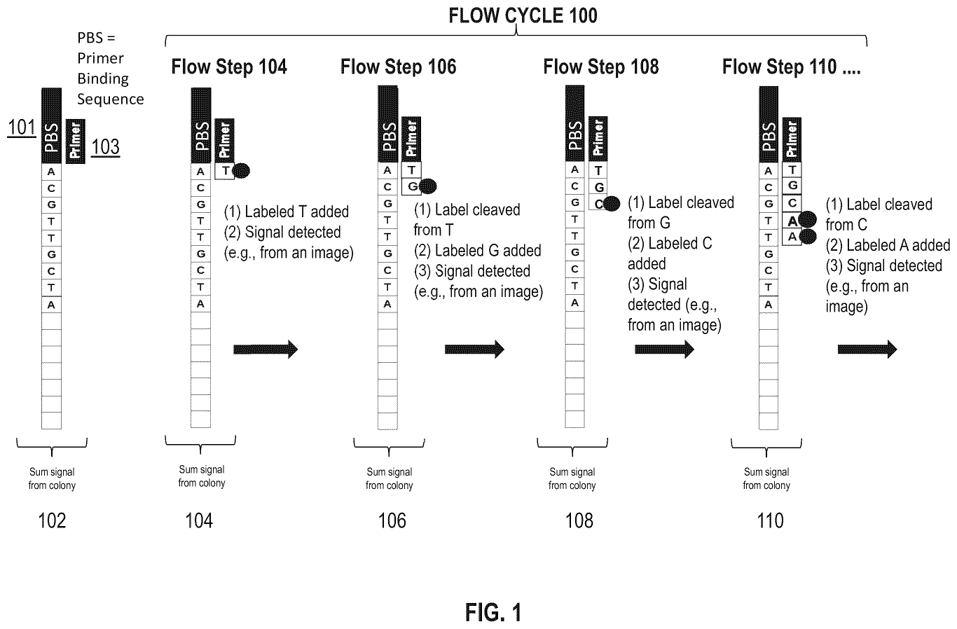

FIG. 1 illustrates an exemplary flow sequencing method that can be used to generate the sequencing data described herein. In some embodiments, polynucleotides may be bound to a surface (e.g., the surface of a bead attached to a substrate), as described in detail herein. The polynucleotides can include a nucleic acid sequence of interest (also referred to as a “template sequence”) and can further include a sequencing adapter sequence. The nucleic acid sequence of interest can be a nucleic acid molecule from or derived from a sample of a subject.

In the depicted example in FIG. 1, the nucleic acid sequence of interest includes a primer binding site (“PBS,” sequence 101) followed by the nucleic acid sequence of interest (e.g., “ACGTTGCTA”).

The adapter sequence 101 can include a sequencing primer hybridization site. At step 102, a sequencing primer 103 is hybridized to the adapter sequence 101 of the polynucleotide at the sequencing primer hybridization site.

The sequencing primer is then extended in a series of flow cycles. In a flow cycle, the hybrid (i.e., the polynucleotide adapter hybridized to the sequencing primer) is combined with nucleotides (e.g., at least partially labeled nucleotides) and one or more signals indicating nucleotide incorporation into the sequencing primer may be detected. In the depicted example, the flow cycle 100 includes four flow steps 104, 106, 108, and 110. In a given flow step, a single type of nucleobase is combined with the hybrid according to the flow-cycle order T-G-C-A. As shown in FIG. 1, in flow step 104, labeled T nucleotides are combined with the hybrid; in flow step 106, labeled G nucleotides are combined with the hybrid; in flow step 108, labeled C nucleotides are combined with the hybrid; in flow step 110, labeled A nucleotides are combined with the hybrid.

At 104, labeled T nucleotides are combined with the hybrid. Since the T base is complementary to the A base in the template polynucleotide, it is incorporated into the extending primer to form the hybrid as shown in 104. Further, a signal indicative of the incorporation of labeled T nucleotide into the sequencing primer can be detected. The signal may be detected, for example, by imaging the surface the polynucleotides are deposited on and analyzing the resulting image(s). In some embodiments, the sequencing platform may be washed with a wash buffer to remove unincorporated nucleotides prior to signal detection. In some embodiments, the detection of the signal is based on image processing techniques described herein.

At step 106, the label may be removed from the T nucleotide (e.g., by cleaving the label from the nucleotide). The sequencing method can then be continued with the next base in the flow order, G in the example illustrated in FIG. 1. At step 106, labeled G nucleotides are combined with the hybrid. Since the G base is complementary to the C base in the template polynucleotide, it is incorporated to form the hybrid in 106. Further, a signal indicating the incorporation of the labeled G nucleotide can be detected.

At step 108, the label may be removed from the G nucleotide (e.g., by cleaving the label from the nucleotide). The sequencing method can then be continued with the next base in the flow order, C. At step 108, labeled C nucleotides are combined with the hybrid. Since the C base is complementary to the G base in the template polynucleotide, it is incorporated into the extending primer to form the hybrid in 108. Further, a signal indicating the incorporation of the labeled C nucleotide into the sequencing primer can be detected.

At step 110, the label may be removed from the C nucleotide (e.g., by cleaving the label from the nucleotide). The sequencing method can then be continued with the next base in the flow order, A. At step 110, labeled A nucleotides are combined with the hybrid. Since the A base is complementary to the T base in the template polynucleotide, it is incorporated into the extending primer to form the hybrid in 110. Further, a signal indicating the incorporation of the labeled A nucleotide into the sequencing primer can be detected.

In step 110, because the template sequence includes two consecutive T bases, two A nucleotides are incorporated into the extending sequencing primer. Thus, the detected signal intensity indicating the incorporation of two A nucleotides may be greater than the signal intensity indicating the incorporation of one nucleotide.

While each flow step in the exemplary flow sequencing method in FIG. 1 results in incorporation of one or more nucleotides (and thus a detected signal indicating such incorporation), it should be appreciated that not all flow steps result in incorporation of nucleotides. In some flow steps, no nucleotide base may be incorporated (for example, in the absence of a complementary base in the template polynucleotide). For example, if C nucleotides are combined with a hybrid having a C base, no incorporation would occur and thus no signal indicative of an incorporation would be detected. Further, as shown in step 110, two nucleotides or more than two nucleotides may be incorporated into the sequencing primer for larger homopolymer lengths in the nucleic acid sequence of interest.

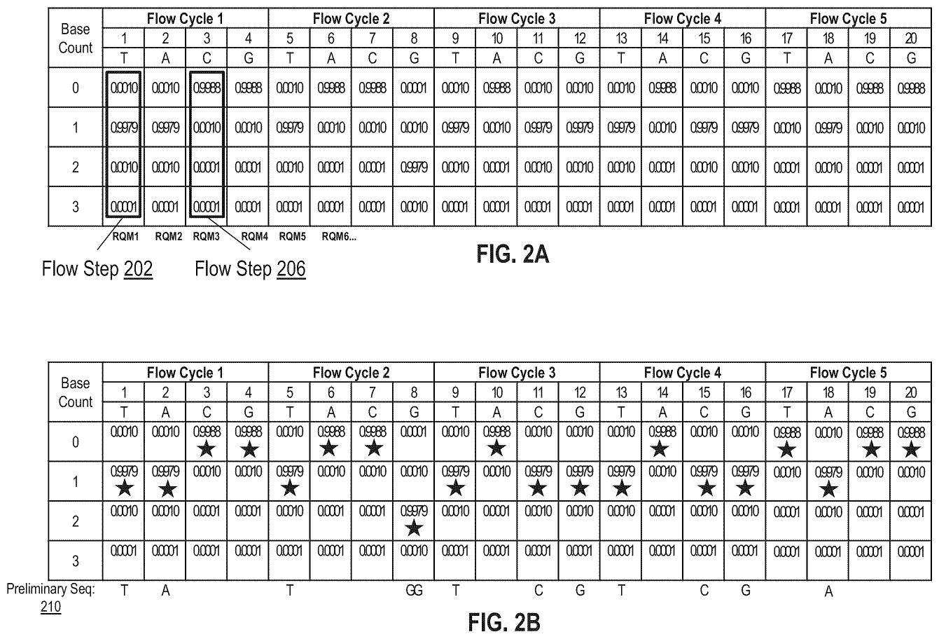

FIG. 2A illustrates an exemplary summary of detected signals after five exemplary flow cycles are performed, in accordance with some embodiments. Solely by way of example, a primer extended using a repeating flow-cycle order of T-A-C-G may result in a sequencing data flowgram set shown in FIG. 2A. Each column in FIG. 2A corresponds to a flow step and the values in each column collectively represent the detected signal intensity in the corresponding flow step, as described below.

In each flow step, the flow signal can be determined from an analog signal that is detected during the sequencing process, such as a fluorescent signal of the one or more bases incorporated into the sequencing primer during sequencing. Although an integer number of zero or more bases are incorporated at any given flow position, a given analog signal many not perfectly match with the analog signal. Therefore, in some embodiments, for a given flow step (e.g., flow step 202), the detected signal intensity can be expressed in probabilistic terms.

Specifically, the detected signal intensity can be expressed in four likelihood values corresponding to 0 base, 1 base, 2 bases, and 3 bases, respectively.

In the depicted example, for flow step 202, the detected signal intensity is expressed by a first likelihood value of 0.001 for 0 base, a second likelihood value of 0.9979 for 1 base, a third likelihood value of 0.001 for 3 bases, and a fourth likelihood value of 0.0001 for 4 bases. This can be interpreted to indicate that there is a high statistical likelihood that one nucleotide base has been incorporated. In the depicted example, the incorporation is a T since the flow step introduced labeled T nucleotides, which means there is an A in the template.

On the other hand, in flow step 206, the detected signal intensity is expressed by a first likelihood value of 0.9988 for 0 base, a second likelihood value of 0.001 for 1 base, a third likelihood value of 0.001 for 3 bases, and a fourth likelihood value of 0.0001 for 4 bases. This can be interpreted to indicate that there is a high likelihood that no nucleotide base has been incorporated. In the depicted example, no C has been incorporated.

Accordingly, the flowgram set in FIG. 2A is formatted as a sparse matrix, with a flow signal represented by a plurality of likelihood values indicating a plurality of likelihoods for a plurality of base homopolymer length counts (e.g., 0 base count, 1 base count, 2 base counts, and 3 base counts) at each flow position.

The homopolymer length likelihood may vary, for example, based on the noise or other artifacts present during detection of the analog signal during sequencing. In some embodiments, if the homopolymer length likelihood statistical parameter or likelihood is below a predetermined threshold, the parameter may be set to a predetermined non-zero value that is substantially zero (i.e., some very small value or negligible value) to aid the downstream statistical analysis further discussed herein, wherein a true zero value may give rise to a computational error or insufficiently differentiate between levels of unlikelihood, e.g., very unlikely (0.0001) and inconceivable (0).

With reference to FIG. 2B, a preliminary sequence can be determined based on the flowgram in FIG. 2A. For example, the most likely sequence can be determined by selecting the base count with the highest likelihood at each flow position, as shown by the stars in FIG. 2B. Thus, the preliminary sequence 210 can be determined as: TATGGTCGTCGA (SEQ ID NO: 1).

From the preliminary sequence (e.g., preliminary sequence 210), the reverse complement (i.e., the template strand or the nucleic acid sequence of interest) can be readily determined. Further, the likelihood of this sequencing data set, given the TATGGTCGTCGA (SEQ ID NO: 1) sequence (or the reverse complement), can be determined as the product of the selected likelihood at each flow position.

The signal for any flow position in the sequencing data is flow-order-dependent in that the flow order used to sequence the polynucleotide at any base position can affect the flow signal at that position. Random fragmentation of nucleic acid molecules (either in vivo fragmentation, such as cell-free DNA, or in vitro fragmentation, such as by sonication or enzymatic digestion) that overlap at the same locus results in multiple different sequencing start sites (relative to the locus) for the nucleic acid molecules.

Sequencing data, such as a flowgram, is based on the detection of a signal detected from an incorporated nucleotide and the order of nucleotide introduction. Take, for example, the flowing template sequences: CTG and CAG, and a repeating flow cycle of T-A-C-G (that is, sequential addition of T, A, C, and G nucleotides, each of which would be incorporated into the primer only if a complementary base is present in the template polynucleotide). A resulting exemplary flowgram is shown in Table 1, where 1 indicates incorporation of an introduced nucleotide and 0 indicates no incorporation of an introduced nucleotide. The flowgram can be used to determine the sequence of the template strand.

TABLE 1

| | Cycle 1 | | | | Cycle 2 | | | |
|---|---|---|---|---|---|---|---|---|
| Sequence | T | A | C | G | T | A | C | G |
| CTG | 0 | 0 | 0 | 1 | 0 | 1 | 1 | 0 |
| CAG | 0 | 0 | 0 | 1 | 1 | 0 | 1 | 0 |
| CCG | 0 | 0 | 0 | 2 | 0 | 0 | 1 | 0 |

The flowgram can be used to quantitatively determine a number of incorporated nucleotides from each stepwise introduction (e.g., for each nucleotide in a cycle). For example, a sequence of CCG would first incorporate two G bases, and any signal emitted by the labeled two bases would have a greater intensity as compared with the incorporation of a single base. This is shown in Table 1 (e.g., the 2 value in the third row). The flowgram of Table 1 indicates the presence or absence of each indicated base, but flowgrams can also provide additional information including the number of bases incorporated at the given step.

Prior to generating the sequencing data, the polynucleotide is hybridized at a hybridization site to a sequencing primer to generate a hybridized template. The polynucleotide may be ligated to an adapter during sequencing library preparation, such as during the attachment of one or more barcode regions. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. patent Ser. No. 10,344,328 and International patent application WO 2020/227143, each of which is incorporated herein by reference in its entirety.

The primer hybridized to the polynucleotide is extended through the nucleic acid molecule using the separate nucleotide flows according to the flow order (which may be cyclical according to a flow-cycle order), and incorporation of a nucleotide can be detected as described above, thereby generating the sequencing data set (via a flowgram) for the nucleic acid molecule.

Alignment (or mapping) of determined sequences to candidate sequences (such as candidate haplotype sequences) in base space is computationally expensive and is currently the most computationally intensive step in, for example, the Genome Analysis Tool Kit (GATK) HaplotypeCaller. Within HaplotypeCaller, PairHMM aligns each sequencing read to each haplotype, and uses base qualities as an estimate of the error to determine the likelihood of the haplotypes given the sequencing read. However, the structure of the data set used with the methods described herein retains error mode likelihoods, which makes variant calling more computationally efficient. For example, a given genotype likelihood may be determined simply as the product of likelihoods in each flow position that aligns with the sequence having the genotype. The flowspace determined likelihood can replace the PairHMM module of the HaplotypeCaller, thus enabling more computationally efficient variant calling.

Primer extension using flow sequencing allows for long-range sequencing on the order of hundreds or even thousands of bases in length. The number of flow steps or cycles can be increased or decreased to obtain the desired sequencing length. Extension of the primer can include one or more flow steps for stepwise extension of the primer using nucleotides having one or more different base types. In some embodiments, extension of the primer includes between 1 and about 1000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, or between about 500 and about 1000 flow steps. The flow steps may be segmented into identical or different flow cycles. The number of bases incorporated into the primer depends on the sequence of the sequenced region, and the flow order used to extend the primer. In some embodiments, the sequenced region is about 1 base to about 4000 bases in length, such as about 1 base to about 10 bases in length, about 10 bases to about 20 bases in length, about 20 bases to about 50 bases in length, about 50 bases to about 100 bases in length, about 100 bases to about 250 bases in length, about 250 bases to about 500 bases in length, about 500 bases to about 1000 bases in length, about 1000 bases to about 2000 bases in length, or about 2000 bases to about 4000 bases in length.

The polynucleotides used in the methods described herein may be obtained from any suitable biological source, for example a tissue sample, a blood sample, a plasma sample, a saliva sample, a fecal sample, or a urine sample. The polynucleotides may be DNA or RNA polynucleotides. In some embodiments, RNA polynucleotides are reverse transcribed into DNA polynucleotides prior to hybridizing the polynucleotide to the sequencing primer. In some embodiments, the polynucleotide is a cell-free DNA (cfDNA), such as a circulating tumor DNA (ctDNA) or a fetal cell-free DNA. The nucleic acid molecules may be randomly fragmented, for example in vivo (e.g., as in cfDNA) or in vitro (for example, by sonication or enzymatic fragmentation).

Libraries of the polynucleotides may be prepared through known methods. In some embodiments, the polynucleotides may be ligated to an adapter sequence. The adapter sequence may include a hybridization sequence that hybridized to the primer extended during the generated of the coupled sequencing read pair.

In some embodiments, the sequencing data is obtained without amplifying the nucleic acid molecules prior to establishing sequencing colonies (also referred to as sequencing clusters). Methods for generating sequencing colonies include bridge amplification or emulsion PCR. Methods that rely on shotgun sequencing and calling a consensus sequence generally label nucleic acid molecules using unique molecular identifiers (UMIs) and amplify the nucleic acid molecules to generate numerous copies of the same nucleic acid molecules that are independently sequenced. The amplified nucleic acid molecules can then be attached to a surface and bridge amplified to generate sequencing clusters that are independently sequenced. The UMIs can then be used to associate the independently sequenced nucleic acid molecules. However, the amplification process can introduce errors into the nucleic acid molecules, for example due to the limited fidelity of the DNA polymerase. In some embodiments, the nucleic acid molecules are not amplified prior to amplification to generate colonies for obtaining sequencing data. In some embodiments, the nucleic acid sequencing data is obtained without the use of unique molecular identifiers (UMIs).

### Exemplary Techniques for Improving Sequencing Read Quality

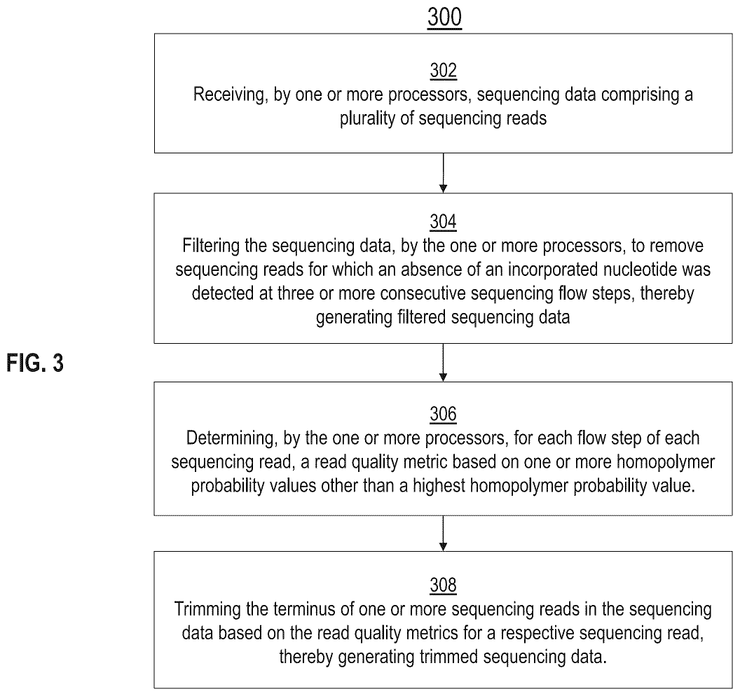

FIG. 3 illustrates an exemplary method 300 for increasing sequencing read quality, in accordance with some embodiments. In some embodiments, process 300 is performed, for example, using one or more electronic devices implementing a software platform. In some examples, process 300 is performed using a client-server system, and the blocks of process 300 are divided up in any manner between the server and client device(s). In other examples, process 300 is performed using only a client device or only multiple client devices. In process 300, some blocks are, optionally, combined, the order of some blocks is, optionally, changed, and some blocks are, optionally, omitted. In some examples, additional steps may be performed in combination with the process 300. Accordingly, the operations as illustrated (and described in greater detail below) are exemplary by nature and, as such, should not be viewed as limiting.

At block 302, an exemplary system (e.g., one or more electronic devices) receives, by one or more processors, sequencing data comprising a plurality of sequencing reads. Each sequencing read of the plurality of sequencing reads can be generated according to a flow sequencing method. As discussed above with reference to FIG. 1, each sequencing read can be generated by extending a sequencing primer (e.g., primer 103) through a region of interest in a target nucleic acid molecule using a plurality of sequencing flow steps (e.g., flow steps 104, 106, 108, 110). Each sequencing flow step can involve combining a hybrid, which comprises the sequencing primer and a nucleic acid molecule comprising the region of interest, with nucleotides, as shown in each of flow steps 104, 106, 108, and 110. At least a portion of the nucleotides are labeled (e.g., T in flow step 104). At each flow step, the presence or absence of an incorporated nucleotide can be detected, and a sequencing read can be generated based on the signals detected over the flow steps, as described with reference to FIGS. 2A-2B. In some embodiments, the nucleotides are non-terminating nucleotides.

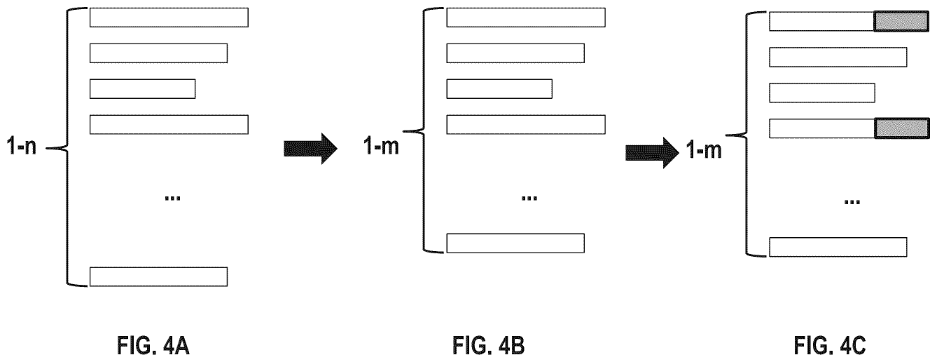

FIG. 4A illustrates an exemplary plurality of sequencing reads that can be received at block 302 of FIG. 3. In FIG. 4A, the system receives n number of sequencing reads. Each sequencing read is obtained from a flow sequencing method. In some embodiments, the sequencing reads are generated by performing one flow sequencing method on a plurality of sequencing colonies attached to the same surface, where each sequencing read corresponds to a sequencing colony. In some embodiments, the sequencing reads are generated by performing multiple flow sequencing methods. The quality of the plurality of sequencing reads can be improved in blocks 304-308, as described below.

At block 304, the system filters the sequencing data, by the one or more processors, to remove sequencing reads for which an absence of an incorporated nucleotide was detected at three or more consecutive sequencing flow steps, thereby generating filtered sequencing data. Specifically, the system can examine each sequencing read of the plurality of sequencing reads one by one to determine if each sequencing read needs to be filtered (i.e., excluded). For each sequencing read, the system determines if the sequencing read indicates an absence of an incorporated nucleotide at three or more consecutive sequencing flow steps, for example, if the sequencing read indicates three consecutive sequencing flow steps yielding no signals (“000”), four consecutive sequencing flow steps yielding no signals (“0000”), five consecutive sequencing flow steps yielding no signals (“00000”), and so on. If this is so, the sequencing read is excluded from the plurality of sequencing reads. That is, the entire length of each sequencing read is evaluated with respect to a number of consecutive sequencing flow steps. With reference to FIG. 4B, the system can examine each of the sequencing reads 1−n and exclude any sequencing read indicating an absence of an incorporated nucleotide at three or more consecutive sequencing flow steps, thus obtaining sequencing reads 1−m (where m<n).

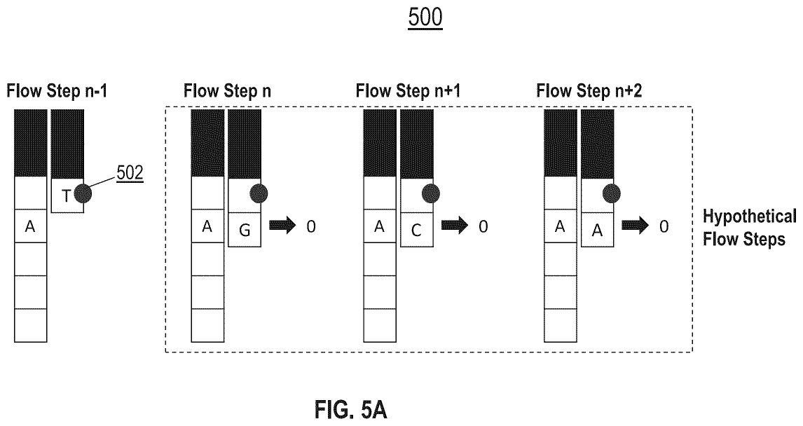

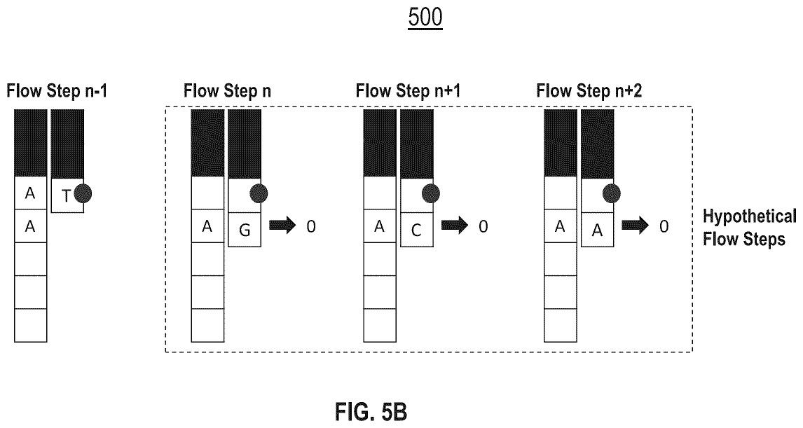

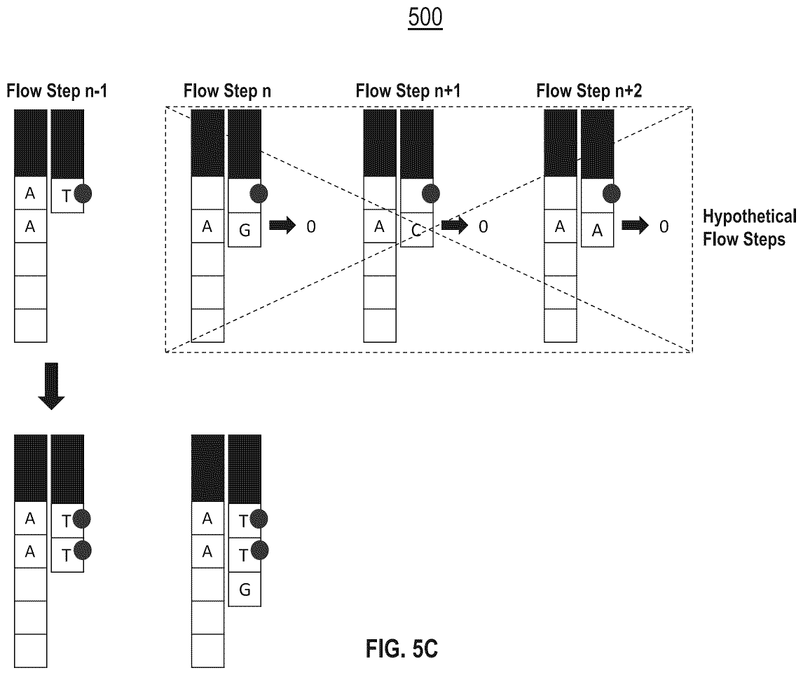

An absence of an incorporated nucleotide at three or more consecutive sequencing flow steps is indicative of weak, incorrect, or noisy signal(s) in the flow sequencing method, and thus an unreliable or damaged sequencing read. FIGS. 5A-5C illustrate an exemplary scenario demonstrating why an absence of an incorporated nucleotide at three consecutive sequencing flow steps cannot occur in a normal sequence. In the flow sequencing method 500, the flow-cycle order is T-G-C-A. In some embodiments, the flow-cycle order is e.g., T-C-G-A, T-A-G-C, or any other permutation of the nucleotides T (or U), G, C, and A. Using an exemplary flow-cycle order T-G-C-A, in flow step n−1, labeled T nucleotides are combined with the hybrid; in flow step n, labeled G nucleotides are combined with the hybrid; in flow step n+1, labeled C nucleotides are combined with the hybrid; in flow step n+2, labeled A nucleotides are combined with the hybrid.

FIG. 5A depicts an impossible hypothetical scenario in which three consecutive sequencing flow steps, n to n+2, all yield a signal of 0 indicating an absence of an incorporated nucleotide. Specifically, in flow step n, labeled G nucleotides are not combined with the hybrid due to the A base; in flow step n+1, labeled C nucleotides are not combined with the hybrid due to the C base; in flow step n+2, labeled A nucleotides are not combined with the hybrid due to the A base.

For the hypothetical scenario in FIG. 5A to occur, there must be a nucleotide incorporation in step n−1 as shown by 502. This is because if there is no nucleotide incorporation in step n−1, in step n, nucleotides G would be combined with the hybrid having the base before A, rather than the hybrid having the base A.

For nucleotide incorporation to occur in step n−1 where labeled T nucleotides are applied, it follows that the base before A in the template polynucleotide must be A (as the T base is complementary to the A base), as shown in FIG. 5B. However, if the base before A in the template polynucleotide is A, the hypothetical flow sequencing steps n to n+2 would not occur. Rather, as shown in FIG. 5C, when labeled T nucleotides are applied in step n−1, two T nucleotides are incorporated into the extending sequencing primer because the template sequence includes two consecutive A bases. Thus, the flow steps n to n+2 depicted in FIG. 5A would not occur.

Thus, FIGS. 5A-5C demonstrate why an absence of an incorporated nucleotide at three consecutive sequencing flow steps cannot occur in a normal sequence. As shown in FIG. 2B, an absence of an incorporated nucleotide can occur in at most two consecutive sequencing flow steps. An absence of an incorporated nucleotide at three or more consecutive sequencing flow steps is indicative of weak, incorrect, or noisy signal(s) in the flow sequencing method, and thus an unreliable or damaged sequencing read. For example, it may indicate that there was a base in the template sequence that had been missed (e.g., indicative of degradation of the template sequence). Thus, any sequencing read having such an absence is filtered in block 304 such that the sequencing read is not used in downstream tasks (e.g., alignment to a reference genome or portions thereof, for SNP calling, etc.).

At block 306, the system determines, by the one or more processors, for each flow step of each sequencing read, a read quality metric. For example, with reference to FIG. 2A, for each flow step (i.e., each column in the flow gram), a read quality metric (also known as regressed residual) is calculated. For example, for flow step 202, a read quality metric RQM1 is calculated; for flow step 206, RQM3 is calculated.

In some embodiments, the read quality metric for each flow step of each sequencing read is calculated based on a second highest homopolymer probability value (p_{2nd}). For example, in flow step 202 in FIG. 2A, the second highest probably value is 0.0010. In some embodiments, the read quality metric (i.e., rₛ) is calculated as:

rₛ=log₁₀(p_{2nd}/ϵ)10, (1)

Where ϵ is a scaling factor and p_{2nd} is the second highest probability at the flow step (e.g., representing the second most likely h-mer). In some embodiments, c can be set at a value between 1×10⁻² and 1×10⁻⁴.

The read quality metric for a given flow step can be calculated using other techniques. In some embodiments, rather than p_{2nd}, (1−p₁ₛₜ) is used in the formula above. In cases in which p₁ₛₜ+p_{2nd}=1, the two formula variations would yield the same read quality metric. In cases in which p₁ₛₜ+p_{2nd}+p_{3rd}=1, the two formula variations would yield different read quality metrics. In most cases, p_{3rd}, p₄ₜₕ, p₅ₜₕ, etc. are small numbers in comparison with p₁ₛₜ and p_{2nd}. In any such case, p₁ₛₜ+p_{2nd}+ . . . +pₙₜₕ=1.

A higher read quality metric can be indicative a weaker signal. For example, a higher p_{2nd} can indicate a lower p₁ₛₜ. Because the base count associated with p₁ₛₜ is selected a lower p₁ₛₜ can indicate a lower confidence in the selected base count. Thus, the read quality metric is used to determine flows with low confidence, which can indicate deterioration in h-mer determination accuracy, in a sequencing read and determine where (e.g., at which flow) to trim the sequencing read, as described below.

It will be understood that the read quality metric could also be calculated, with appropriate modifications to the read quality metric function, using any h-mer probability value each flow step of each sequencing read (e.g., p₁ₛₜ, p_{2nd}, p_{3rd} . . . , pₙₜₕ). Calculating the read quality metric with, for example, a first highest homopolymer probability value can be performed thus:

rₛ=log₁₀((1−p₁ₛₜ)/ϵ)/10, (2)

where ϵ would be set as in equation (1).

At block 308, the system trims the terminus of one or more sequencing reads in the sequencing data based on the read quality metrics for a respective sequencing read, thereby generating trimmed sequencing data. With reference to FIG. 4C, some of the sequencing reads 1-m are trimmed, thereby generating trimmed sequencing data.

In some embodiments, if a flow sequencing step produces a read quality metric below a predetermined threshold, the system can determine that deterioration has occurred in the sequencing read. Accordingly, the system can trim the sequencing read at or before the first flow sequencing step that produces a read quality metric below the threshold.

In some embodiments, the system uses an average of multiple read quality values to detect determination in the sequencing read. In some embodiments, the average is a moving average. Exemplary calculation of the moving average is described with reference to FIG. 2A. For example, at the third flow step, the system can calculate an average of RQM1, RQM2, and RQM3 (assuming the moving average is calculated using a sliding window of 3 flow steps); at the fourth flow step, the system can calculate an average of RQM2, RQM3, and RQM4. Thus, the moving average is a local quality measure.

In some embodiments, if the moving average exceeds a predetermined threshold, the system determines that deterioration (e.g., of read quality) has occurred and trims the sequencing read accordingly. In some embodiments, if a predefined number of moving averages are above the predetermined threshold, the system determines that deterioration has occurred. For example, the flow sequencing step that triggers trimming is the nth sequencing flow step having a moving average above a predetermined threshold, wherein n is a predefined number. That is, in some instances, the sequencing read is trimmed at the flow where the read quality moving average exceeds the predetermined threshold. In some instances, the sequencing read is trimmed at the nth-flow where the read quality moving average has exceeded the predetermined threshold. In some instances, trimming the sequencing read removes the indicated flow and all subsequent flows.

The predetermined threshold can be a fixed value that can be tuned. For example, the predetermined threshold can be set to an average quality of the first 100 flow steps in a flow sequencing method (e.g., based on an average read quality metric for each flow across all sequencing reads). In some embodiments, the predetermined threshold is around 0.3. In some embodiments, the predetermined threshold is about 0, 0.1, 0.2, 0.3, 0.4, or 0.5. In some embodiments, the predetermined threshold is a real number between any of 0, 0.1, 0.2, 0.3, 0.4, or 0.5. Likewise, the predetermined number n can be a tunable fixed value. For example, n can be set to 3, 5, 10, 15, or 20. In some instances, n is any whole number between 1 and 20.

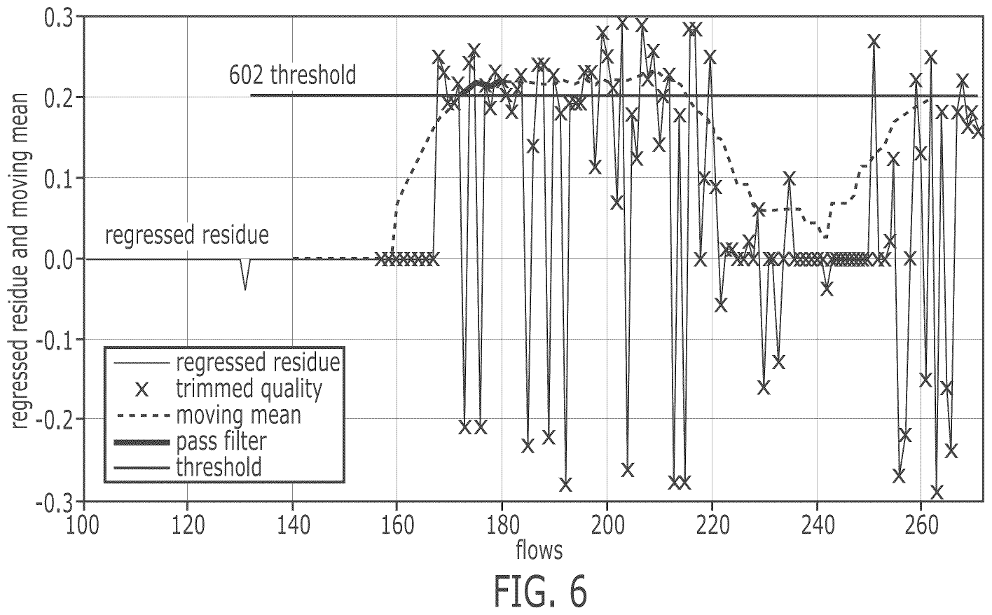

FIG. 6 illustrates the read quality metrics for an exemplary sequencing read, in accordance with some embodiments. In the depicted example, each cross indicates the read quality metric calculated at the corresponding flow step. The dashed line indicates the moving average of read quality metrics. The horizontal line 602 indicates the predetermined threshold (i.e., 0.2). If a predefined number of consecutive moving averages exceed the predetermined threshold (as shown by the bolded portion of the dashed line above the line 602), the system determines that deterioration has occurred and therefore trims the sequencing read.

The system then trims at least the portion of the sequencing read comprising the selected sequencing flow step. In some embodiments, a predetermined number of consecutive sequencing flow steps prior to the selected sequencing flow step are also trimmed. In some embodiments, the predetermined number of consecutive sequencing flow steps is a multiple of four (e.g., 8 previous flow steps, 12 previous flow steps, 16 previous flow steps). In other words, the system also trims multiples of 4 flow steps before the selected flow step, in addition to trimming the selected flow step.

Thus, the trimming operation in block 308 can be dependent on at least three parameters: window length, threshold, and lag. Window length refers to the size of the sliding window in which the moving average value is calculated. Threshold refers to the predetermined threshold of the moving average value above which the system determines that deterioration has occurred. Lag refers to the predetermined number of consecutive sequencing flow steps prior to the selected sequencing flow step that are also trimmed. In some embodiments, some or all of these parameters can be determined based on user input. In some embodiments, some or all of these parameters can be determined automatically.

In some embodiments, the system does not calculate a read quality metric for every flow step, but rather at regular intervals (e.g., every 4 flow steps, every 8 flow steps, etc.). In some embodiments, these regular intervals will be a multiple 4. In some embodiments, the system does not calculate read quality metrics for certain flow steps in a flow sequencing method (e.g., the first 100 flow sequencing steps), for example because deterioration typically occurs during later flow steps.

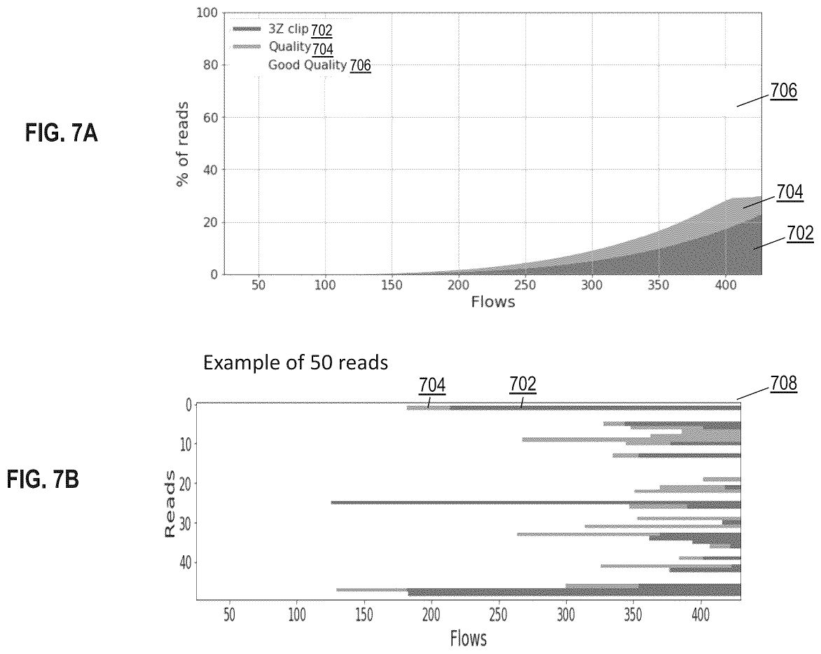

FIG. 7A illustrates that quality issues may occur to an increasing percentage of reads as the number of flow steps increases. As shown by the area 702, as the number of flow steps increases, a higher percentage of sequencing reads are filtered (referred to as “3Z clip”) due to the absence of an incorporated nucleotide at three or more consecutive sequencing flow steps in these sequencing reads. As shown in the area 704, as the number of flow steps increases, a higher percentage of sequencing reads are trimmed based on read quality metric calculations (referred to as “Quality”). For example, at flow step 350, about 10% of reads are removed and <10% of reads are trimmed. At flow step 400, about 30% of the sequencing reads have quality issues and are either trimmed or removed, and about 70% of the sequencing reads do not have quality issues (as shown in area 706).

FIG. 7B illustrates 50 exemplary sequencing reads in accordance with some embodiments. The 50 sequencing reads are represented by 50 horizontal lines. Every line starts with a white segment, indicating that no quality issues have been detected. In some of the reads, quality issues are eventually detected. For example, in read 708, around flow step 180, an absence of an incorporated nucleotide at three or more consecutive sequencing flow steps is detected. At around flow step 220, deterioration is detected based on read quality metrics. If the method 300 in FIG. 3 is performed to process the 50 reads, any of the 50 reads that has an absence of an incorporated nucleotide at three or more consecutive sequencing flow steps (i.e., any read having the segment of the shading 702) would be filtered in block 304; any of the remaining reads that have deterioration based on the read quality metric (i.e., any read having a segment of the shading 704 but not a segment of shading 702) would be trimmed in block 308 but still included in the downstream tasks.

In some embodiments, the system trims a known adapter sequence, or portion thereof, from one or more sequencing reads in the sequencing data. Sequencing adapters (e.g., the primer binding site sequence 101 in FIG. 1) can be ligated to the ends of the individual nucleic acids. The adapters serve as binding sites for primers (e.g., primer 103 in FIG. 1). It can be beneficial to trim the adapters because they can increase the file size (e.g., the CRAM file size) but are not useful for downstream tasks. Trimming the adapters can improve data quality (e.g., for variant calling) while reducing the size of output files.

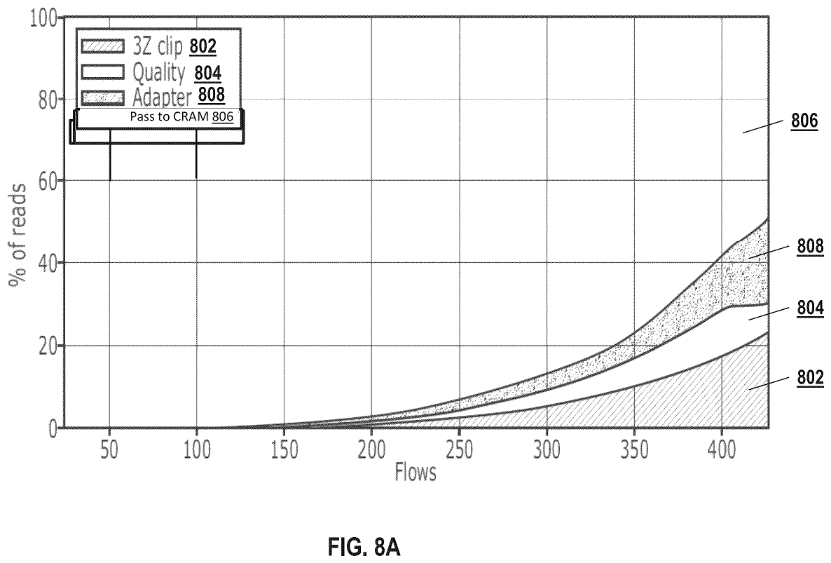

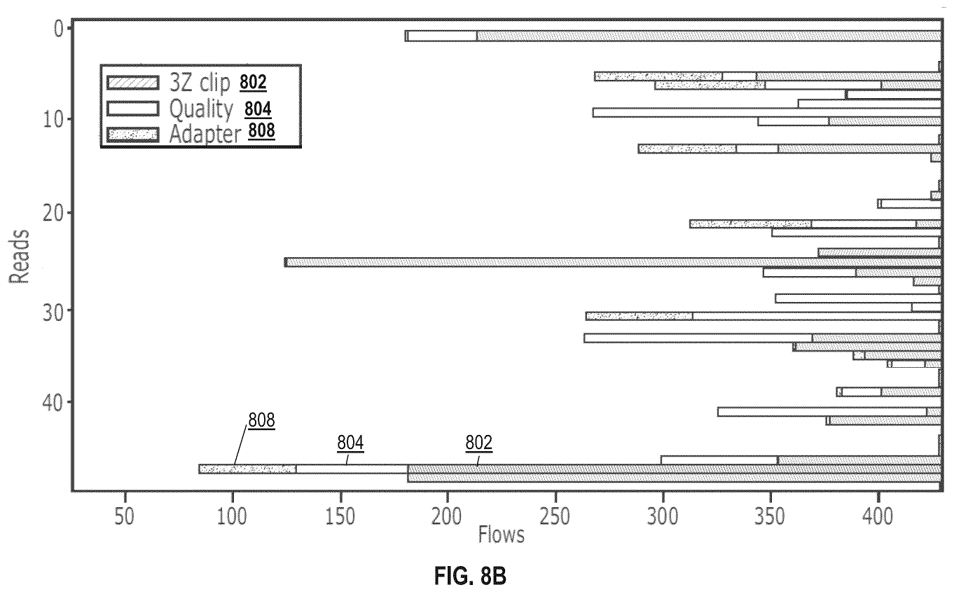

In some embodiments, the identification and trimming of adapters are performed after trimming in block 308. FIG. 8A illustrates that quality issues may occur to an increasing percentage of reads as the number of flow steps increases. In the depicted example, the percentage of reads with trimmed adapters 808 increases as the number of flow steps increases. This may be because the adapter sequences are at the opposite end of reads from the primer locations where the sequencing begins. Thus, adapter sequences are only observed (and then trimmed) in later flows. Overall sequencing read quality, and downstream analysis, is improved by the removal of adapter sequences. This is because adapter sequences cannot be accurately aligned to a reference sequence (e.g., because adapter sequences are synthetic and are not expected to exist in a reference sequence); thus, if a read includes residual adapter sequence, misalignments may occur during alignment processes, which may decrease accuracy in downstream variant calling. FIG. 8B illustrates 50 exemplary sequencing reads in accordance with some embodiments. The 50 sequencing reads are represented by 50 horizontal lines. The segments of the shading 808 indicate reads that are trimmed due to adapter identification.

In some embodiments, the system stores the trimmed sequencing data in a non-transitory computer readable medium.

In some embodiments, the system aligns sequencing reads in the trimmed sequencing data to a reference sequence (e.g., for variant calling). The method 300 improves the quality of the sequencing reads (e.g., by removing undesirable reads and/or trimming undesirable portions of reads). The resulting sequencing reads are more likely to be aligned to the reference genome. In some embodiments, at least a predetermined percentage of sequencing reads in the trimmed sequencing data are aligned to the reference sequence. In some embodiments, the predetermined percentage is about 70%, about 75%, about 80%, about 85%, about 90%, about 95%, about 99%, or about 100%. In some embodiments, the system calls, using the one or more processors, one or more genetic variants using the trimmed sequencing data set (e.g., by aligning or otherwise comparing the trimmed sequencing data to a reference sequence such as a reference genome). In some embodiments, the method 300 is agnostic in terms of nucleotide data and thus can be used for RNA and/or DNA.

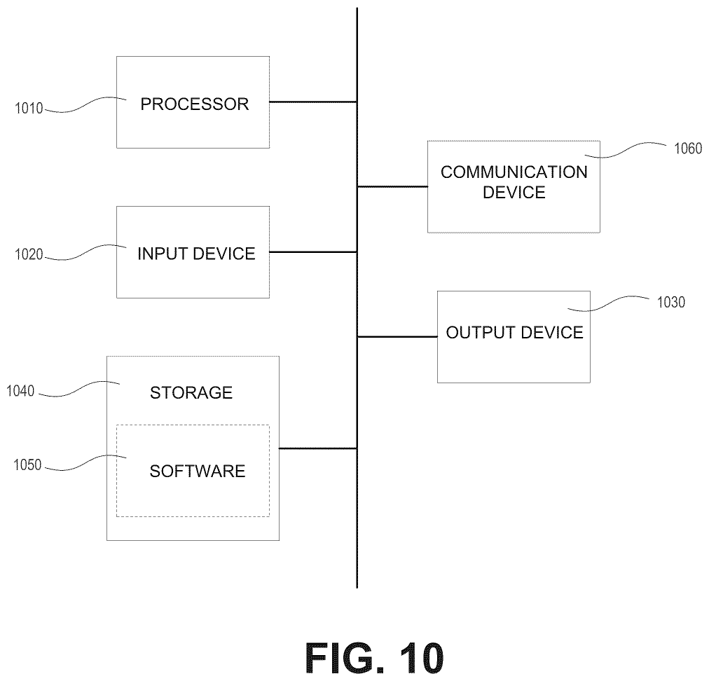

Some or all operations described herein with reference to FIGS. 1-9 are optionally implemented by components depicted in FIG. 10. FIG. 10 illustrates an example of a computing device in accordance with some embodiments. Device 1000 can be a host computer connected to a network. Device 1000 can be a client computer or a server. As shown in FIG. 10, device 1000 can be any suitable type of microprocessor-based device, such as a personal computer, workstation, server or handheld computing device (portable electronic device) such as a phone or tablet. The device can include, for example, one or more of processors 1010, input device(s) 1020, output device(s) 1030, storage 1040, and communication device(s) 1060.

Input device 1020 and output device 1030 can be either connectable to or integrated with device 1000. Input device 1020 can be any suitable device that provides input, such as a touch screen, keyboard or keypad, mouse, or voice-recognition device. Output device 1030 can be any suitable device that provides output, such as a screen, touch screen, haptics device, or speaker.

Storage 1040 can be any suitable device that provides storage, such as an electrical, magnetic or optical memory including a RAM, cache, hard drive, or removable storage disk. Storage 1040 encompasses persistent memory and non-persistent memory. Non-persistent memory includes electronically addressable solid-state memory and mechanically addressable memory (e.g., hard disks, optical disks, tape, etc.). In some embodiments, non-persistent memory includes high-speed random-access memory or other random-access solid-state memory devices. Persistent memory optionally includes one or more remote storage devices (e.g., remote from the one or more processors). In some embodiments, persistent memory and/or non-volatile memory device(s) within non-persistent memory comprises non-transitory computer readable storage medium.

Communication device 1060 can include any suitable device capable of transmitting and receiving signals over a network, such as a network interface chip or device. The components of the computer can be connected in any suitable manner, such as via a physical bus or wirelessly. In some embodiments, communication device 1060 includes communication buses, including circuitry that interconnects and controls communications between device 1000 components.

Software 1050, which can be stored in storage 1040 and executed by processor 1010, can include, for example, the programming that embodies the functionality of the present disclosure (e.g., as embodied in the devices as described above). Software 1050 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1040, that can contain or store programming for use by or in connection with an instruction execution system, apparatus, or device. Software 1050 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 1000 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1000 can implement any operating system suitable for operating on the network. Software 1050 can be written in any suitable programming language, such as C, C++, Java, Python, etc. In various embodiments, application software embodying the functionality of the present disclosure can be deployed in different configurations, such as in a client/server arrangement or through a Web browser as a Web-based application or Web service, for example.

As described with respect to FIG. 10, device 1000 can store and process sequencing read data in accordance with methods described herein. Specifically, memory 1040 (e.g., a non-transitory computer readable medium) may store the following information:

An operating system, including procedures for handling various basic system services and for performing hardware-dependent tasks;

A trimming module including instructions for trimming sequencing reads as described herein;

A filtering module including instructions for filtering sequencing reads as described herein;

An input data set comprising sequencing information for a first plurality of sequencing reads;

A filtered data set comprising sequencing information for a second plurality of sequencing reads, where the second plurality of sequencing reads is a subset of the first plurality of sequencing reads, where the subset has the same or less than the total number of sequencing reads in the first plurality of sequencing reads;

One or more trimmed data sets comprising sequencing information for the second plurality of sequencing reads, where one or more sequencing reads in the second plurality of sequencing reads has been trimmed in accordance with one or more trimming methods described herein;

An optional network communication module, or instructions, for connecting the device 1000 with other devices or a communication network;

An I/O module including procedures for handling various basic input and output functions through the input and output devices (1020, 1030); and

Optionally, additional modules including instructions for handling other functions and aspects described herein.

In some embodiments, one or more of the above-mentioned elements is stored in memory as described above. The above-mentioned elements each correspond to a set of instructions for a function as described above. The above-mentioned modules, data, or programs may be implemented as separate software programs, procedure, datasets, or modules. Alternatively, or in addition, the above-mentioned modules, data, or programs may be combined or otherwise rearranged in various implementations.

Although FIG. 10 depicts device 1000, this is intended as a functional description of the various features that may be present in a device rather than as a structural schematic of the implementations described herein. As will be recognized by those of skill in the art, items that are shown as combined may be separated, and some items may be combined.

### Exemplary Data Structures

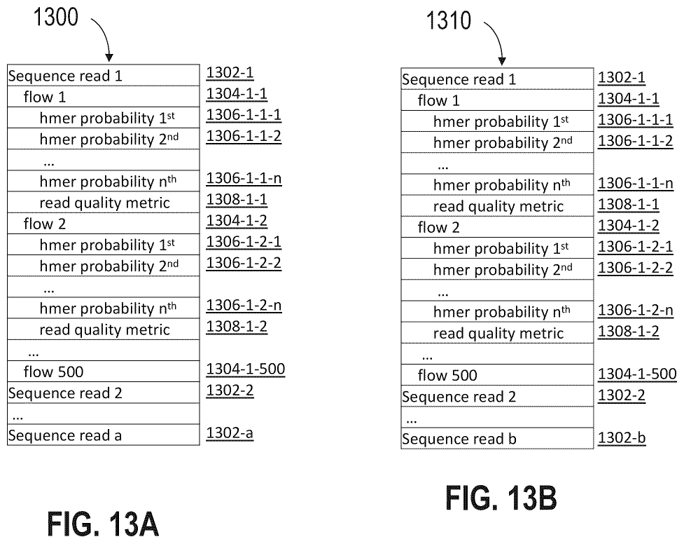

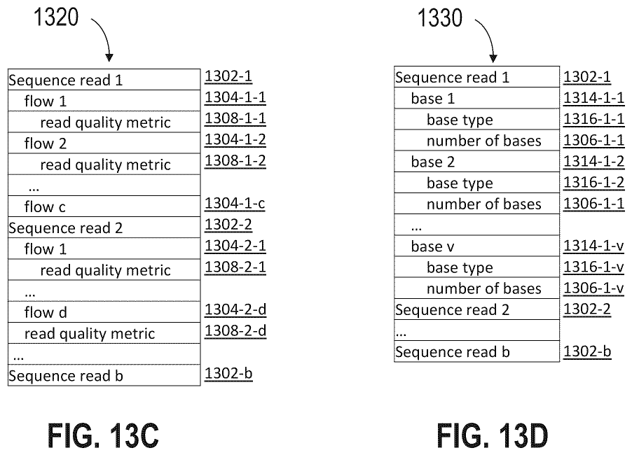

While methods in accordance with the present disclosure have been disclosed above, more details as to the types of data that may be processed or provided by these methods are now described. FIGS. 13A-13D illustrate example block diagrams of sequencing read data sets in accordance with embodiments described herein.

FIG. 13A shows an example of an input sequencing read data set. An input data set 1300 (e.g., comprising flow-based signal information for a first plurality of sequencing reads) may include, for each sequencing read 1302 in the first plurality of sequencing reads, for each sequencing flow 1304 in a plurality of sequencing flows, at least: i) at least two homopolymer (hmer) probabilities 1306 (e.g., each hmer probability represents a likelihood of a particular number (h) of consecutive nucleic acids of a single base type being detected for a respective flow), and ii) a read quality metric 1308, where the read quality metric is determined from the hmer probabilities. The first plurality of sequencing reads comprises a number a of sequencing reads. In this example input data set 1300, the total number of sequencing flows is 500. As described elsewhere herein, a total number of sequencing flows may be any desired number. In some embodiments, an hmer probability is computed for each sequencing read, for each sequencing flow, for each value of h from 0-12. In some embodiments, an hmer probability is computed for each sequencing read, for each sequencing flow, for each value of h from 0-11, from 0-10, from 0-9, from 0-8, from 0-7, from 0-6, from 0-5, or from 0-4.

FIG. 13B shows an example of a filtered sequencing read data set. A filtered data set 1310 may include, for each sequencing read 1302 in the second plurality of sequencing reads, for each sequencing flow 1304 in a plurality of sequencing flows, at least: i) at least two homopolymer (hmer) probabilities 1306 (e.g., each hmer probability represents a likelihood of a particular number (h) of consecutive nucleic acids of a single base type being detected for a respective flow), and ii) a read quality metric 1308, where the read quality metric is determined from the hmer probabilities. The second plurality of sequencing reads comprises a number b of sequencing reads that are a subset from the first plurality of sequencing reads, where b is less than or equal to a. The second plurality of sequencing reads are filtered from the first plurality of sequencing read, where the filtering removes sequencing reads that exhibit the absence of an incorporated nucleotide at three or more consecutive sequencing flow steps in these sequencing reads (e.g., as described herein with regards to FIGS. 5A-5C). In some embodiments, the absence of an incorporated nucleotide is determined based on hmer probabilities (e.g., for sequencing reads with three consecutive sequencing flows that each have a highest hmer probability for h=0).

FIG. 13C shows an example of a trimmed sequencing read data set. Trimmed data set 1320 may include, for each sequencing read in the second plurality of sequencing reads

FIG. 13D shows another example of a trimmed sequencing read data set. Trimmed data set 1330 may include, for each sequencing read in the second plurality of sequencing reads, for each base call 1314, at least: (i) a nucleotide base type 1316, and ii) a number of nucleotide bases 1318, where the number of nucleotide bases is determined from the hmer probabilities 1306 (e.g., for each flow with at least one non-zero hmer probability or at least one hmer probability above a base call threshold), and the nucleotide base type is determined based on the known flow order (e.g., the base type corresponding to each flow in the plurality of flows is known). This trimmed data set 1330 illustrates example data produced as a result of adaptor trimming and is performed after base calling. As will be understood to one of skill in the art, other variants of trimmed data sets may also be provided by the methods described herein.

In some embodiments, trimming and filtering actions may be performed in any order. For example, in some embodiments, read filtering (e.g., 3Z clipping) may be performed prior to read quality trimming. In some other embodiments, read quality trimming may be performed prior to read filtering. In some other embodiments, base calling is performed after read quality trimming. In some embodiments, base calling (e.g., converting from flowspace to basespace for a sequencing read) is performed prior to read quality trimming. In some embodiments, base calling is performed prior to adaptor trimming. In some embodiments, adaptor trimming is performed after read quality trimming.

### EXAMPLES

### Example 1: Impact of Read Trimming on Read Length Distribution

Sequence information using 500 sequencing flows as described above was collected for about 1×10⁹ sequencing reads. A random sample of 20×10⁶ sequencing reads were analyzed based on read quality, as described herein. This is performed to evaluate the overall quality of a sequencing run (e.g., the series of 500 sequencing flows). The read trimming is performed on the entirety of the 1×10⁹ sequencing reads obtained.

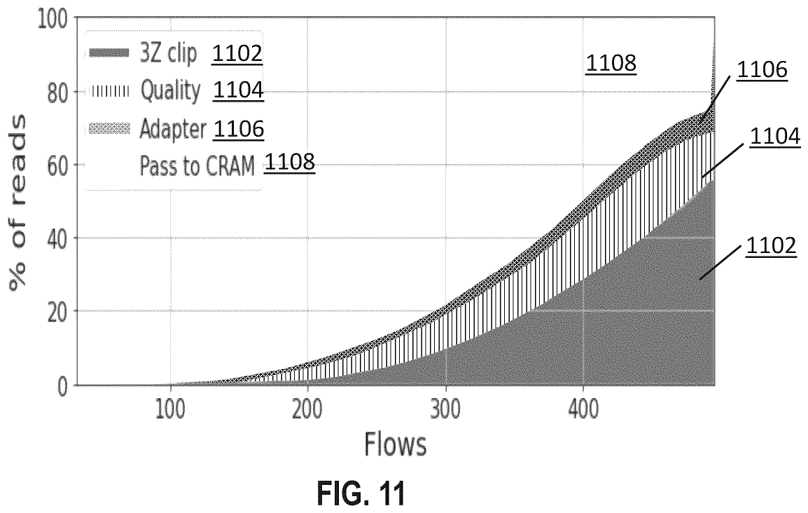

FIG. 11 illustrates the percentages of the randomly sampled sequencing reads that were filtered in accordance with different quality metrics. The overall quality of sequencing reads in FIG. 11 decreases as the number of flow steps increases. Area 1102 shows that, as the number of flow steps increases, a higher percentage of sequencing reads are filtered due to the absence of an incorporated nucleotide at three or more consecutive sequencing flow steps in these sequencing reads (i.e., 3Z clipping). Area 1104 shows that, as the number of flow steps increases, a higher percentage of sequencing reads are trimmed based on read quality metrics. Area 1106 shows that, as the number of flow steps increases, a higher percentage of sequencing reads are trimmed to remove adapter sequence. The remaining sequencing reads, as indicated by area 1108, are not trimmed (e.g., ‘passed to CRAM’ as is). At flow 500, the end of the sequencing run, about 70% of sequencing reads have been trimmed or filtered as a result of quality degradation and an increase in 3 consecutive 0 signal flows, respectively. An additional approximately 15% of sequencing reads have adapter sequence that is trimmed by flow 500.

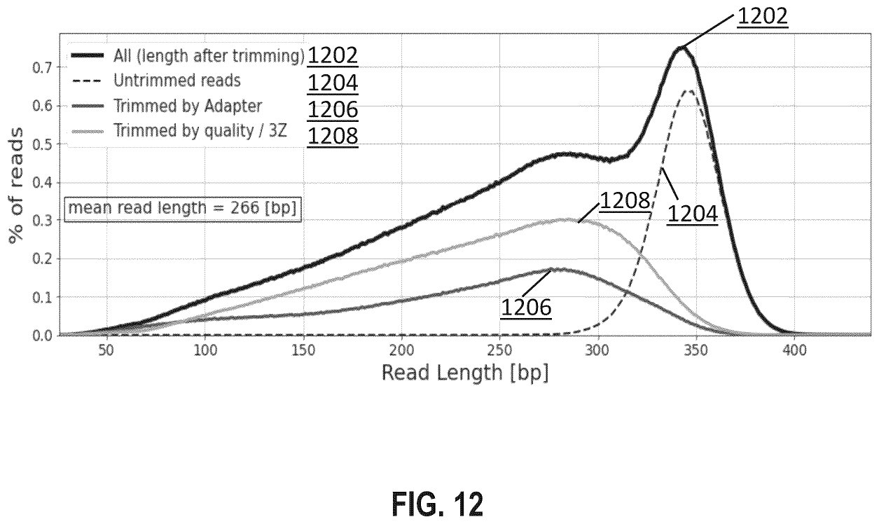

FIG. 12 illustrates the read length in number of bases (bp) for sequencing reads in the randomly sampled set of sequencing reads at various stages of read trimming/filtering. One result of trimming sequencing reads is a decrease in average read length for all sequencing reads in a run. For example, in FIG. 12 the mode of read length for all reads after trimming/filtering is about 340 base pairs, and the mode of read length for all untrimmed reads is about 345 base pairs. The mean read length for all reads after trimming/filtering is about 266 base pairs. The increase in overall read quality offsets any disadvantages that may arise from performing downstream analysis (e.g., variant determination) with shorter reads.

### Example 2—Read Trimming Benefits

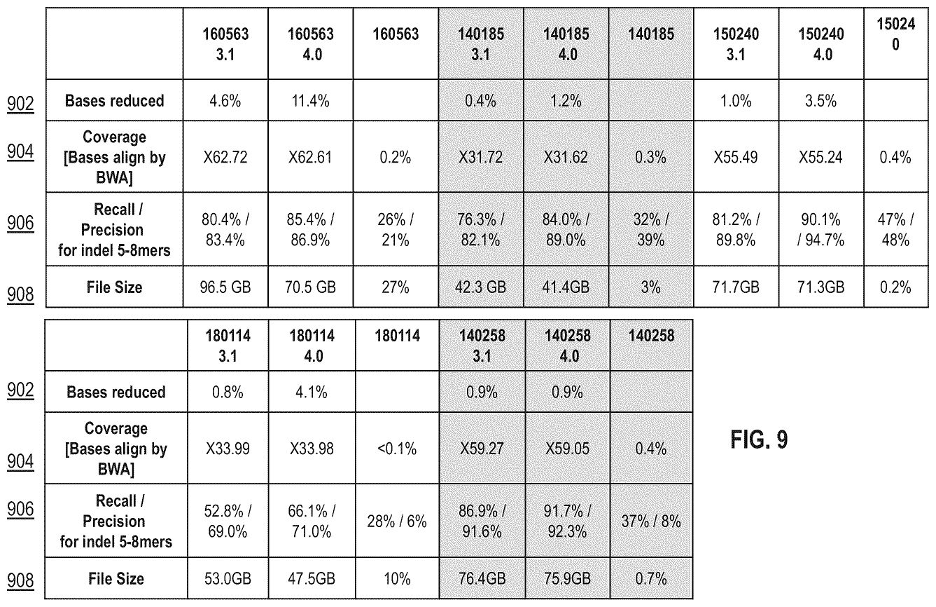

FIG. 9 illustrates exemplary results of for various sequencing runs, in accordance with some embodiments of methods described herein. Results from five sequencing runs, identified as 160563, 140185, 150240, 180114, and 140258, are provided. For each sequencing run, two versions of the method 300 are performed on the sequencing reads of the sequencing run.

Version 3.1 refers to a version of the method including filtering to remove sequencing reads for which an absence of an incorporated nucleotide was detected at three or more consecutive sequencing flow steps, but excluding trimming based on a read quality metric. Version 4.0 refers to a version including both filtering and trimming steps.

As shown in row 902, both Version 3.1 and Version 4.0 lead to a reduction of bases in each sequencing run (i.e., a removal of problematic reads and/or portions of reads), thus improving the overall quality of the sequencing reads available for downstream analysis. While Version 4.0 results in additional bases reduced as shown in row 902, row 904 shows that, advantageously, Version 4.0 does not significantly reduce coverage. For example, for sequencing run 160563, the coverage of the sequencing reads after Version 3.1 is performed is 62.72 times, and the coverage of the sequencing reads after Version 4.0 is performed is 62.61 times, leading to only a 0.2% reduction in coverage. Thus, the resulting coverages of the two versions do not significantly differ.

Row 906 shows the recall and precision metrics for Versions 3.1 and 4 for each sequencing run. Precision and recall are two metrics which together are used to evaluate the performance of an exemplary variant calling system. Precision can be defined as the fraction of relevant instances (i.e., true positives) among all retrieved instances (i.e., true positives and false positives). Recall, also referred to as sensitivity, can be defined as the fraction of retrieved instances (i.e., true positives) among all relevant instances (i.e., true positives and false negatives). In row 906, the improvement value of the recall metric is shown in the third column for every sequencing run and is calculated as: 1−((1−recall value for version 4.0/100)÷(1−recall value for version 3.1/100)). The improvement value of the precision metric is calculated similarly. The improvement values represent the reduction in undetected (or misdirected) variants. As shown, Version 4.01 results in improved performance of variant calling.

Row 908 shows the resulting file sizes (e.g., of CRAM files) for Versions 3.1 and 4.0 for each sequencing run. In each sequencing run, Version 4.0 results a larger reduction of the file size. Thus, the method results in a smaller file of sequencing reads for downstream tasks (e.g., variant calling). The smaller files advantageously require less computer storage space, thus leading to improved usage and management of computer memory. The smaller files can be faster to process in downstream tasks, resulting in a more efficient use of computer processing power. Further, the smaller files contain cleaner, better-structured data, thus improving the analysis capability of downstream tasks, as illustrated by row 906. Thus, embodiments of the present disclosure improve the functioning of computer systems and sequencing systems. Through novel data structures and logics, embodiments of the present disclosure provide improved memory usage, improved memory management, and improved processing to support the high-throughput and high-precision requirements of the flow sequencing method to provide high-quality sequencing reads.

Although the disclosure and examples have been fully described with reference to the accompanying FIGURES, it is to be noted that various changes and modifications will become apparent to those skilled in the art. Such changes and modifications are to be understood as being included within the scope of the disclosure and examples as defined by the claims.

The foregoing description and examples are for purpose of explanation and have been detailed with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

What is claimed is:

1. A method for increasing sequencing read quality, comprising:
receiving, at one or more processors, sequencing data comprising a plurality of sequencing reads generated by extending a sequencing primer through a region of interest in a target nucleic acid molecule using a plurality of sequencing flow steps, each sequencing flow step comprising combining a hybrid with nucleotides, the hybrid comprising the sequencing primer and a nucleic acid molecule comprising the region of interest, wherein at least a portion of the nucleotides are labeled, and detecting the presence or absence of an incorporated nucleotide;
filtering the sequencing data, using the one or more processors, to remove sequencing reads for which an absence of an incorporated nucleotide was detected at three or more consecutive sequencing flow steps, thereby generating filtered sequencing data;
determining, using the one or more processors, for each sequencing flow step of each sequencing read, a read quality metric based on one or more homopolymer probability values other than a highest homopolymer probability value; and
trimming the terminus of one or more sequencing reads in the sequencing data based on the read quality metrics for a respective sequencing read, thereby generating trimmed sequencing data.

2. The method of claim 1, comprising generating the sequencing data.

3. The method of claim 1 or 2, comprising calling, using the one or more processors, one or more genetic variants using the trimmed sequencing data.

4. The method of any one of claims 1-3, further comprising trimming a known adapter sequence, or a portion thereof, from one or more sequencing reads in the sequencing data.

5. The method of any one of claims 1-3, wherein the read quality metric for each sequencing flow step of each sequencing read is based on a second highest homopolymer probability value.

6. The method of any one of claims 1-5, wherein trimming the terminus of the one or more sequencing reads in the sequencing data based on the read quality metric, thereby generating the trimmed sequencing data, comprises, for each sequencing read:
determining a read quality metric moving average for the sequencing flow steps;
selecting a sequencing flow step, wherein the selected sequencing flow step is the nth sequencing flow step having a moving average above a predetermined threshold, wherein n is a predefined number; and
trimming at least a portion of the sequencing read comprising the selected sequencing flow step.

7. The method of claim 6, wherein a predetermined number of consecutive sequencing flow steps prior to the selected sequencing flow step are trimmed.

8. The method of claim 7, wherein the predetermined number of consecutive sequencing flow steps is a multiple of four.

9. The method of any one of claims 1-8, further comprising storing the trimmed sequencing data in a non-transitory computer readable medium.

10. The method of any one of claims 1-9, further comprising aligning sequencing reads in the trimmed sequencing data to a reference sequence.

11. The method of claim 10, wherein at least a predetermined percentage of sequencing reads in the trimmed sequencing data are aligned to the reference sequence.

12. The method of claim 11, wherein the predetermined percentage is about 70%, about 75%, about 80%, about 85%, about 90%, about 95%, about 99%, or about 100%.

13. The method of any one of claims 10-12, wherein the reference sequence is a reference genome.

14. The method of any one of claims 1-13, wherein the nucleotides are non-terminating nucleotides.

15. A system, comprising:
one or more processors; and
a non-transitory computer readable medium storing one or more programs which, when executed by the one or more processors, are configured to:
receive, at the one or more processors, sequencing data comprising a plurality of sequencing reads generated by extending a sequencing primer through a region of interest using a plurality of sequencing flow steps, each sequencing flow step comprising combining a hybrid with nucleotides, the hybrid comprising the sequencing primer and a nucleic acid molecule comprising the region of interest, wherein at least a portion of the nucleotides are labeled, and detecting the presence or absence of an incorporated nucleotide;
filter the sequencing data, using the one or more processors, to remove sequencing reads for which an absence of an incorporated nucleotide was detected at three or more consecutive sequencing flow steps, thereby generating filtered sequencing data;
determine, using the one or more processors, for each flow step of each sequencing read, a read quality metric based on one or more homopolymer probability values other than a highest homopolymer probability value; and
trim the terminus of one or more sequencing reads in the sequencing data based on the read quality metrics for a respective sequencing read, thereby generating trimmed sequencing data.

16. The system of claim 15, further comprising a sequencer configured to generate the sequencing data.

17. The system of claim 15 or 16, wherein the one or more programs, when executed by the one or more processors, are further configured to call, using the one or more processors, one or more genetic variants using the trimmed sequencing data.

18. The system of any one of claims 15-17, wherein the one or more programs, when executed by the one or more processors, are further configured to trim a known adapter sequence, or a portion thereof, from one or more sequencing reads in the sequencing data.

19. The system of any one of claims 15-18, wherein the read quality metric for each sequencing flow step of each sequencing read is based on a second highest homopolymer probability value.

20. The system of any one of claims 15-18, wherein trimming the terminus of the one or more sequencing reads in the sequencing data based on the read quality metric, thereby generating the trimmed sequencing data, comprises, for each sequencing read:
determining a read quality metric moving average for the sequencing flow steps;
selecting a sequencing flow step, wherein the selected sequencing flow step is the nth sequencing flow step having a moving average above a predetermined threshold, wherein n is a predetermined number; and
trimming at least a portion of the sequencing read comprising the selected sequencing flow step.

21. The system of claim 20, wherein a predetermined number of consecutive sequencing flow steps prior to the selected sequencing flow step are trimmed.

22. The system of claim 21, wherein the predetermined number of consecutive sequencing flow steps is a multiple of four.

23. The system of any one of claims 15-22, wherein the one or more programs, when executed by the one or more processors, are further configured to store the trimmed sequencing data in the non-transitory computer readable medium.

24. The system of any one of claims 15-23, wherein the one or more programs, when executed by the one or more processors, are further configured to align sequencing reads in the trimmed sequencing data to a reference sequence.

25. The system of claim 24, wherein at least a predetermined percentage of sequencing reads in the trimmed sequencing data are aligned to the reference sequence.

26. The system of claim 25, wherein the predetermined percentage is about 70%, about 75%, about 80%, about 85%, about 90%, about 95%, about 99%, or about 100%.

27. The system of any one of claims 15-26, wherein the nucleotides are non-terminating nucleotides.
